(19) United States
(12) Patent Application Publication
(10) Pub. No.: US 2020/0214679 A1
(43) Pub. Date: Jul. 9, 2020

(54) METHODS AND APPARATUSES FOR RECEIVING FEEDBACK FROM USERS REGARDING AUTOMATIC CALCULATIONS PERFORMED ON ULTRASOUND DATA

(71) Applicants: Nathan Silberman, Brooklyn, NY (US); Tomer Gafner, Forest Hills, NY (US)

(72) Inventors: Nathan Silberman, Brooklyn, NY (US); Tomer Gafner, Forest Hills, NY (US)

(73) Assignee: Butterfly Network, Inc., Guilford, CT (US)

(21) Appl. No.: 16/733,742

(22) Filed: Jan. 3, 2020

Related U.S. Application Data
(60) Provisional application No. 62/788,698, filed on Jan. 4, 2019.

(51) Int. Cl. A61B 8/08

(52) U.S. Cl. CPC A61B 8/5207; A61B 8/462; A61B 8/5292

(57) ABSTRACT

Aspects of the technology described herein relate to techniques for receiving feedback from a user regarding an automatic calculation performed based on ultrasound data. The automatic calculation may be a result for a measurement performed on the ultrasound data. The feedback from the user may include an indication of agreement or disagreement with the result of the measurement; an indication of whether the result of the measurement is too high, too low, or correct; a result for the measurement that the user considers to be correct; or locations on one or more ultrasound images where one or more statistical models should have focused when performing the automatic calculation. The automatic calculation may also include a quality of the ultrasound data for performing a measurement, and the feedback may include an indication whether the user considers the ultrasound data acceptable for performing the measurement or not.

### CROSS-REFERENCE TO RELATED APPLICATIONS

The present application claims the benefit under 35 U.S.C. § 119(e) of U.S. Patent Application Ser. No. 62/788,698, filed Jan. 4, 2019 under Attorney Docket No. B1348.70124US00, and entitled “METHODS AND APPARATUSES FOR RECEIVING FEEDBACK FROM USERS REGARDING AUTOMATIC CALCULATIONS PERFORMED ON ULTRASOUND DATA,” which is hereby incorporated herein by reference in its entirety.

### FIELD

Generally, the aspects of the technology described herein relate to receiving feedback from users regarding automatic calculations performed on ultrasound data.

### BACKGROUND

Ultrasound probes may be used to perform diagnostic imaging and/or treatment, using sound waves with frequencies that are higher than those audible to humans. Ultrasound imaging may be used to see internal soft tissue body structures. When pulses of ultrasound are transmitted into tissue, sound waves of different amplitudes may be reflected back towards the probe at different tissue interfaces. These reflected sound waves may then be recorded and displayed as an image to the operator. The strength (amplitude) of the sound signal and the time it takes for the wave to travel through the body may provide information used to produce the ultrasound image. Many different types of images can be formed using ultrasound devices. For example, images can be generated that show two-dimensional cross-sections of tissue, blood flow, motion of tissue over time, the location of blood, the presence of specific molecules, the stiffness of tissue, or the anatomy of a three-dimensional region.

### SUMMARY

According to one aspect, an apparatus comprises a processing device in operative communication with an ultrasound device and configured to receive feedback from a user regarding an automatic calculation performed based on ultrasound data.

In some embodiments, the processing device is further configured to perform the automatic calculation based on the ultrasound data. In some embodiments, the processing device is configured, when performing the automatic calculation, to use one or more statistical models. In some embodiments, the processing device is further configured to receive the ultrasound data from the ultrasound device.

In some embodiments, the automatic calculation based on the ultrasound data comprises a result for a measurement performed automatically on the ultrasound data. In some embodiments, the processing device is configured, when receiving the feedback from the user, to receive an indication of agreement or disagreement with the result of the measurement. In some embodiments, the processing device is configured, when receiving the feedback from the user, to receive an indication of whether the result of the measurement is too high, too low, or correct. In some embodiments, the processing device is configured, when receiving the feedback from the user, to receive a value for the measurement that the user considers to be correct. In some embodiments, the processing device is configured, when receiving the feedback from the user, to receive one or more locations on one or more ultrasound images where one or more statistical models should have focused when performing the measurement. In some embodiments, the processing device is configured, when receiving the feedback from the user, to receive a flag to review the ultrasound data and/or the result of the measurement performed automatically.

In some embodiments, the automatic calculation based on the ultrasound data comprises a quality of the ultrasound data determined automatically for performing a measurement on the ultrasound data. In some embodiments, the processing device is configured, when receiving the feedback from the user, to receive an indication whether the user considers the ultrasound data acceptable for performing the measurement or not. In some embodiments, the processing device is configured, when receiving the feedback from the user, to receive a flag to review the ultrasound data and/or the quality of the ultrasound data determined automatically.

In some embodiments, the processing device is configured, when receiving the feedback, to receive text from the user. In some embodiments, the processing device is further configured to provide an option that the user may select to provide the feedback. In some embodiments, the processing device is further configured to display an image produced from the ultrasound data.

In some embodiments, the processing device is further configured to upload the ultrasound data used for the automatic calculation, the automatic calculation, and the feedback to one or more servers. In some embodiments, the one or more servers are configured to train one or more statistical models to more accurately perform the automatic calculation based on the ultrasound data used for the automatic calculation, the automatic calculation, and the feedback. In some embodiments, the processing device is further configured to download the one or more statistical models from the one or more servers. In some embodiments, the processing device is further configured to train one or more statistical models to more accurately perform the automatic calculation based on the ultrasound data used for the automatic calculation, the automatic calculation, and the feedback.

Some aspects include at least one non-transitory computer-readable storage medium storing processor-executable instructions that, when executed by at least one processor, cause the at least one processor to perform the functions of the above apparatus. Some aspects include a method for performing the functions of the above apparatus.

### BRIEF DESCRIPTION OF THE DRAWINGS

Various aspects and embodiments will be described with reference to the following exemplary and non-limiting figures. It should be appreciated that the figures are not necessarily drawn to scale. Items appearing in multiple figures are indicated by the same or a similar reference number in all the figures in which they appear.

FIG. 1 illustrates an example process for receiving feedback from a user regarding automatic calculations performed on ultrasound data, in accordance with certain embodiments described herein;

FIG. 2 illustrates an example process for receiving feedback from a user regarding automatic calculations performed on ultrasound data, in accordance with certain embodiments described herein;

FIG. 3 illustrates an example process for receiving feedback from a user regarding automatic calculations performed on ultrasound data, in accordance with certain embodiments described herein;

FIG. 4 illustrates an example graphical user interface (GUI) that may be shown by the processing device, in accordance with certain embodiments described herein;

FIG. 5 illustrates another example GUI that may be shown by the processing device, in accordance with certain embodiments described herein;

FIG. 6 illustrates another example GUI that may be shown by the processing device, in accordance with certain embodiments described herein;

FIG. 7 illustrates another example GUI that may be shown by the processing device, in accordance with certain embodiments described herein;

FIG. 8 illustrates another example GUI that may be shown by the processing device, in accordance with certain embodiments described herein;

FIG. 9 illustrates another example GUI that may be shown by the processing device, in accordance with certain embodiments described herein;

FIG. 10 illustrates another example GUI that may be shown by the processing device, in accordance with certain embodiments described herein;

FIG. 11 illustrates another example GUI that may be shown by the processing device, in accordance with certain embodiments described herein;

FIG. 12 illustrates another example GUI that may be shown by the processing device, in accordance with certain embodiments described herein;

FIG. 13 illustrates another example GUI that may be shown by the processing device, in accordance with certain embodiments described herein;

FIG. 14 illustrates another example GUI that may be shown by the processing device, in accordance with certain embodiments described herein;

FIG. 15 illustrates another example GUI that may be shown by the processing device, in accordance with certain embodiments described herein;

FIG. 16 illustrates another example GUI that may be shown by the processing device, in accordance with certain embodiments described herein;

FIG. 17 illustrates another example GUI that may be shown by the processing device, in accordance with certain embodiments described herein;

FIG. 18 illustrates another example GUI that may be shown by the processing device, in accordance with certain embodiments described herein;

FIG. 19 illustrates another example GUI that may be shown by the processing device, in accordance with certain embodiments described herein;

FIG. 20 illustrates another example GUI that may be shown by the processing device, in accordance with certain embodiments described herein;

FIG. 21 shows an example of a “virtuous circle” for continuously improving the performance of certain processes and systems described herein, in accordance with certain embodiments described herein;

FIG. 22 shows a schematic block diagram illustrating aspects of an example ultrasound system upon which various aspects of the technology described herein may be practiced;

FIG. 23 shows a schematic block diagram illustrating aspects of another example ultrasound system upon which various aspects of the technology described herein may be practiced; and

FIG. 24 illustrates an example convolutional neural network that is configured to analyze an image.

### DETAILED DESCRIPTION

Advances in artificial intelligence technology have enabled automatic performance of measurements on ultrasound images, potentially obviating the need for operators to have the required knowledge for manually performing such measurements. An ultrasound device may collect an ultrasound image and automatically perform a measurement on the ultrasound image by inputting the image to a statistical model that is trained on training data to automatically perform this measurement. Aspects of such automatic measurements are described in U.S. patent application Ser. No. 15/626,423 titled “AUTOMATIC IMAGE ACQUISITION FOR ASSISTING A USER TO OPERATE AN ULTRASOUND IMAGING DEVICE,” filed on Jun. 19, 2017 (and assigned to the assignee of the instant application) and published as U.S. Pat. Pub. 2017/0360401 A1, which is incorporated by reference herein in its entirety.

One training data point may include an ultrasound image labeled with a measurement that was performed manually on the ultrasound image. Based on this training data, the statistical model may learn to perform measurements automatically when confronted with new ultrasound images. Ideally, the statistical model will perform measurements accurately on images collected from any individual, regardless of the individual's particular characteristics such as age, size, health, etc. In other words, ideally the statistical model will perform measurements accurately across the entire distribution of individuals. This may require that the training data accurately represent the entire distribution of individuals (e.g., across the distribution of ages, sizes, health, etc.). Thus, a problem can arise if an ultrasound device collects an ultrasound image from an individual not adequately represented in the training data, as the statistical model run by the ultrasound device may not perform an accurate measurement on the ultrasound image. While the theoretical solution is to collect training data that represents the entire distribution of individuals at the outset, this may not be practically feasible.

The inventors have implemented a practical solution to this problem, whereby the statistical model running on ultrasound devices is periodically retrained on new training data. In instances where a statistical model does not, in a user's opinion, perform a measurement on an image accurately, this may indicate that the statistical model was not trained on data that accurately represented this particular image. The human may provide feedback on the measurement (e.g., provide a value for the measurement that s/he thinks is accurate). This feedback may be uploaded to a server and used to train a statistical model stored on the server, where the feedback may serve as another training data point that can be used to retrain the statistical model and potentially help the statistical model perform the measurement more accurately on similar images in the future. The retrained statistical model may then be downloaded to many users' ultrasound devices.

It should be noted that an individual ultrasound device may not only improve based on its own user's feedback, but will receive the benefit of feedback received from many other users of other ultrasound devices. In particular, feedback received from many other users of other ultrasound devices may be more likely to be representative of the entire distribution of individuals than feedback received from one user of an individual ultrasound device. For example, one user may have more access to older individuals, while another user may have more access to younger individuals. The accuracy of the statistical model is performing measurements, and the functionality of individual ultrasound devices running the statistical model, may thereby improve based on this access to a wider distribution individuals. Additionally, statistical models may perform measurements more accurately based on training data labeled with manual measurements performed by many users rather than a single user, due to averaging out of idiosyncrasies of individual users. The functioning of an individual ultrasound device may therefore also improve based on access to a wider distribution of users who are providing feedback.

It should be appreciated that the embodiments described herein may be implemented in any of numerous ways. Examples of specific implementations are provided below for illustrative purposes only. It should be appreciated that these embodiments and the features/capabilities provided may be used individually, all together, or in any combination of two or more, as aspects of the technology described herein are not limited in this respect.

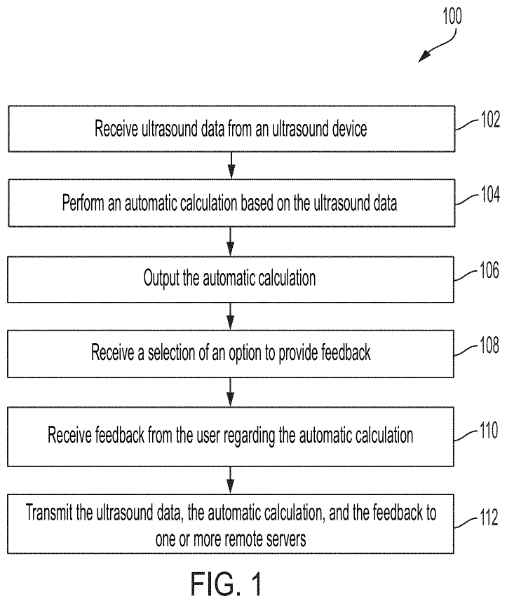

FIG. 1 illustrates an example processes 100 for receiving feedback from a user regarding automatic calculations performed on ultrasound data, in accordance with certain embodiments described herein. The process 100 is performed by a processing device that is part of or in operative communication with an ultrasound device. The processing device may be, for example, a mobile phone, tablet, or laptop. The ultrasound device and the processing device may communicate over a wired communication link (e.g., over Ethernet, a Universal Serial Bus (USB) cable or a Lightning cable) or over a wireless communication link (e.g., over a BLUETOOTH, WiFi, or ZIGBEE wireless communication link).

In act 102, the processing device receives ultrasound data from the ultrasound device. For example, the processing device may receive from the ultrasound device raw acoustical data, scan lines generated from raw acoustical data, and/or one or more ultrasound images generated from raw acoustical data or scan lines. The process 100 proceeds from act 102 to act 104.

In act 104, the processing device performs an automatic calculation based on the ultrasound data. For example, the processing device may perform the automatic calculation using statistical models (either on the processing device itself or by accessing the statistical models on a remote server). The statistical models may include, for example, a convolutional neural network, a deep learning model, a random forest, a support vector machine, or a linear classifier. In some embodiments, the processing device may perform the automatic calculation on the same ultrasound data received from the ultrasound device in act 102. In other embodiments, the processing device may perform the automatic calculation on data generated from the ultrasound data received in act 102. For example, the processing device may perform the automatic calculation on scan lines, an ultrasound image, or multiple ultrasound images generated from the ultrasound data received in act 102. In some embodiments, the automatic calculation may be a measurement performed automatically on one or more ultrasound images. For example, the measurement may include automatic calculation of ejection fraction using Simpson's method by automatic localization in ultrasound images of two keypoints that are base points of the mitral valve. Other examples of measurements include aortic root measurements, fetal measurements, inferior vena cava diameter and compressibility, carotid intima-media thickness, gallbladder wall thickness, tricuspid annular plane systolic excursion, cardiac or carotid velocity time integral, or measurements for detecting abdominal aortic aneurysm, B-lines, kidney stones, pneumonia, appendicitis, carotid plaque, deep vein thrombosis, focal wall motion abnormalities, free fluid in abdomen, hypertrophic cardiomyopathy. For further description of automatically performing measurement, see U.S. patent application Ser. No. 15/626,423 titled “AUTOMATIC IMAGE ACQUISITION FOR ASSISTING A USER TO OPERATE AN ULTRASOUND IMAGING DEVICE,” filed on Jun. 19, 2017 (and assigned to the assignee of the instant application). In some embodiments, the automatic calculation may be a calculation of a quality metric representing the quality of one or more ultrasound images for the purpose of automatically performing a measurement (e.g., for automatic calculation of ejection fraction). In such embodiments, the processing device may output (e.g., display on a display screen) the quality metric (e.g., a value). For further description of the quality indicator, see U.S. patent application Ser. No. 16/172,076 titled “QUALITY INDICATORS FOR COLLECTION OF AND AUTOMATED MEASUREMENT ON ULTRASOUND IMAGES,” filed on Oct. 26, 2018 (and assigned to the assignee of the instant application), which is incorporated by reference herein in its entirety. It should be appreciated that other automatic calculations may also be performed. The process 100 proceeds from act 104 to act 106.

In act 106, the processing device outputs (e.g., displays on a display screen) the automatic calculation. For example, if the automatic calculation is a measurement, the processing device may display the result of the measurement (e.g., a value). As another example, if the automatic calculation is calculation of a quality metric, the processing may display a value for the quality metric. The process 100 proceeds from act 106 to act 108.

In act 108, the processing device receives a selection of an option to provide feedback. For example, the processing device may display the option on a display screen, and the processing device may receive a selection of the option (e.g., through a touch or a click on the option). Upon receiving the selection of the option, the processing device may receive the feedback from the user (as described with reference to act 110). In some embodiments, act 108 may be absent, and the processing device may receive feedback from the user without receiving a selection of an option to provide feedback. The process 100 proceeds from act 108 to act 110.

In act 110, the processing device receives feedback from a user regarding the automatic calculation. In some embodiments, if the processing device outputs a value that is the result of a measurement automatically performed on one or more ultrasound images (e.g., a value for ejection fraction), the processing device may receive feedback from the user indicating agreement or disagreement with the result of the measurement. In some embodiments, the processing device may receive feedback from the user indicating whether the result of the measurement is too high, too low, or correct. In some embodiments, the processing device may receive feedback from the user consisting of the value for the measurement that the user considers to be correct. To receive feedback consisting of a value that the user considers to be the correct result for the measurement, the processing device may display a number pad that the user may use to input the value. In some embodiments, the processing device may receive feedback from the user consisting of locations on the one or more ultrasound images where the statistical models should have focused when automatically performing the measurement. In some embodiments, if the processing device outputs a quality metric representing a quality of one or more ultrasound images for performing a particular measurement, the user may receive from the user feedback consisting of an indication whether the user considers the one or more ultrasound images acceptable for the measurement or not (e.g., “measurable” vs. “not measurable”). To receive feedback consisting of an indication whether the user considers the one or more ultrasound images acceptable for the measurement or not, the processing device may display two buttons, one corresponding to measurable and one corresponding to not measurable. Thus, the user's feedback may agree or disagree with the automatic calculation. In some embodiments, the processing device may receive feedback in the form of text from the user, which may consist of the user's comments. The process 100 proceeds from act 110 to act 112.

In act 112, the processing device transmits the ultrasound data (e.g., one or more ultrasound images) used for the automatic calculation, the automatic calculation (e.g., the result of the measurement performed automatically or the value for the quality metric), and the user's feedback (e.g., the user's value for the measurement, the indication of whether the ultrasound data is measurable or not measurable) to one or more servers (e.g., “the cloud”). In some embodiments, the processing device may upload ultrasound data that is different than the ultrasound data used to perform the automatic calculation. For example, if raw acoustical data or scan lines are used to perform the automatic calculation, the processing device may still upload one or more ultrasound images generated from the raw acoustical data or scan lines. The processing device may transmit data to the one or more remote servers over a wired communication link (e.g., over Ethernet, a Universal Serial Bus (USB) cable or a Lightning cable) or over a wireless communication link (e.g., over a BLUETOOTH, WiFi, or ZIGBEE wireless communication link). This information may be used to train statistical models on the cloud to more accurately perform automatic calculations, such as calculations of quality and measurement values.

For example, if the feedback from the user is agreement with the result of the measurement, then the statistical models may be retrained with new training data including the ultrasound data labeled with the result of the measurement. If the feedback from the user is disagreement with the result of the measurement, then the statistical models may be retrained with new training data including the ultrasound data labeled with a constraint that the measurement is within a certain percentage (e.g., 5%, 10%, 15%, 20%, any value in between, or any other suitable a value) of the result of the measurement. (This constraint assumes that although the user disagrees with the result of the measurement automatically performed by the statistical models, the result of the measurement performed automatically is nevertheless still within a certain percentage of the correct value). If the feedback from the user is that the result of the measurement is too high, then the statistical models may be retrained with new training data including the ultrasound data labeled with a constraint that the measurement is within a range that is a certain percentage (e.g., 5%, 10%, 15%, 20%, any value in between, or any other suitable a value) lower than the result of the measurement. If the feedback from the user is that the result of the measurement is too low, then the statistical models may be retrained with new training data including the ultrasound data labeled with a constraint that the measurement is within a range that is a certain percentage (e.g., 5%, 10%, 15%, 20%, any value in between, or any other suitable a value) greater than the result of the measurement. If the feedback from the user is that the result of the measurement is correct, then the statistical models may be retrained with new training data including the ultrasound data labeled with the result of the measurement. If the feedback from the user consists of the value for the measurement that the user considers to be correct, then the statistical models may be retrained with new training data including the ultrasound data labeled with the value from the user. The retrained statistical models may then be downloaded by the processing device. The retraining and downloading may occur periodically (e.g., every week, every two weeks, every month, or any other suitable frequency).

In some embodiments, the information may be used to train statistical models on the processing device itself. In such embodiments, act 112 may be absent. In some embodiments, an ultrasound device may perform the process 100. In such embodiments, the ultrasound device may include circuitry for performing the automatic calculation and circuitry for transmitting data to remote severs. Additionally, in such embodiments, the act 102 may be absent. In some embodiments, the ultrasound device may perform the act 104, transmit the automatic calculation to the processing device, and the processing device may perform the acts 108, 110, and 112.

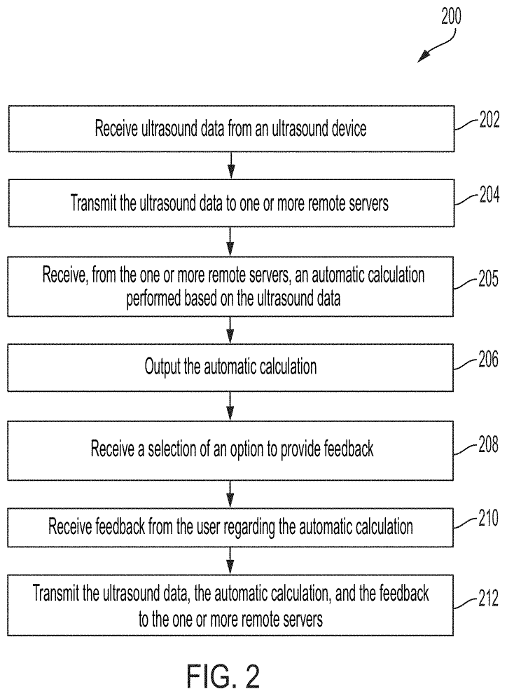

FIG. 2 illustrates another example processes 200 for receiving feedback from a user regarding automatic calculations performed on ultrasound data, in accordance with certain embodiments described herein. As in the process 100, the process 200 is performed by a processing device that is part of or in operative communication with an ultrasound device.

Acts 202, 206, 208, 210, and 212 are the same as acts 102, 106, 108, 110, and 112, respectively. In act 204, the processing device transmits the ultrasound data to one or more servers (e.g., “the cloud”). In some embodiments, the processing device may transmit the same ultrasound data received from the ultrasound device in act 202. In other embodiments, the processing device may transmit data generated from the ultrasound data received in act 202. For example, the processing device may transmit scan lines, an ultrasound image, or multiple ultrasound images generated from the ultrasound data received in act 202. The processing device may transmit data to the one or more remote servers over a wired communication link (e.g., over Ethernet, a Universal Serial Bus (USB) cable or a Lightning cable) or over a wireless communication link (e.g., over a BLUETOOTH, WiFi, or ZIGBEE wireless communication link). The process 200 proceeds from act 204 to act 205.

In act 205, the processing device receives, from the one or more remote servers, an automatic calculation performed based on the ultrasound data. For example, if the automatic calculation is a measurement, the processing device may receive the result of the measurement (e.g., a value) from the one or more remote servers. As another example, if the automatic calculation is calculation of a quality metric, the processing device may receive a value for the quality metric from the one or more remote servers. The process 200 proceeds from act 205 to act 206.

As described with reference to the process 100, in some embodiments act 202, act 206, act 208, and/or act 212 may be absent. Additionally, as described with reference to the process 100, in some embodiments the ultrasound device may perform certain of the acts of the process 200.

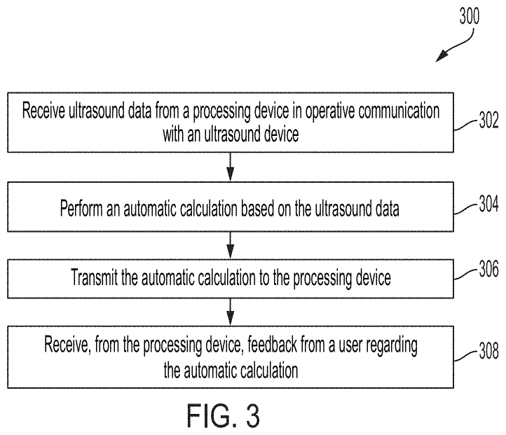

FIG. 3 illustrates another example processes 300 for receiving feedback from a user regarding automatic calculations performed on ultrasound data, in accordance with certain embodiments described herein. The process 300 is performed by one or more remote servers in communication with a processing device, where the processing device is part of or in operative communication with an ultrasound device. The one or more remote servers and the processing device may communicate over a wired communication link (e.g., over Ethernet, a Universal Serial Bus (USB) cable or a Lightning cable) or over a wireless communication link (e.g., over a BLUETOOTH, WiFi, or ZIGBEE wireless communication link).

In act 302, the one or more remote servers receive ultrasound data from a processing device in operative communication with an ultrasound device. For example, the one or more remote servers may receive raw acoustical data, scan lines, an ultrasound image, or multiple ultrasound images from the processing device. The process 300 proceeds from act 302 to act 304.

In act 304, the one or more remote servers performs an automatic calculation based on the ultrasound data. Further description of performing automatic calculations may be found with reference to act 104. The process 300 proceeds from act 304 to act 306.

In act 306, the one or more remote servers transmits an automatic calculation to the processing device. For example, if the automatic calculation is a measurement, the one or more remote servers may transmit the result of the measurement (e.g., a value) to the processing device. As another example, if the automatic calculation is calculation of a quality metric, the one or more remote servers may transmit a value for the quality metric to the processing device. The process 300 proceeds from act 306 to act 308.

In act 308, the one or more remote servers receive, from the processing device, feedback from a user regarding the automatic calculation. The processing device may receive the feedback from the user, as described with reference to act 110, and transmit the feedback to the one or more remote servers.

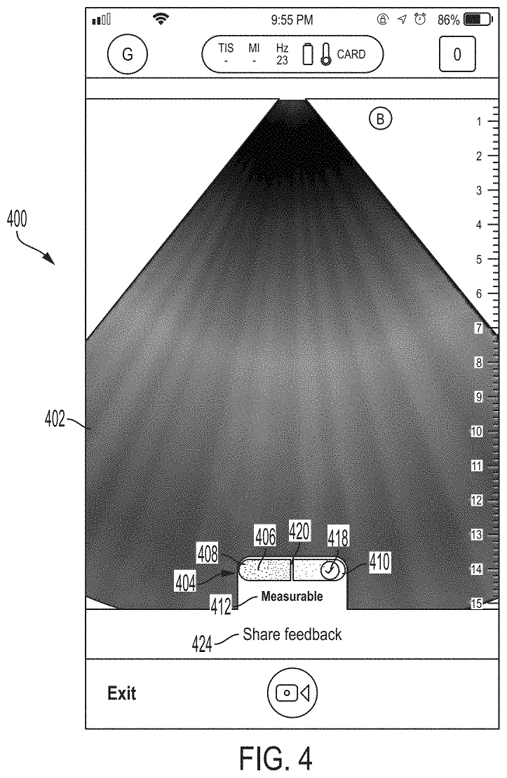

FIGS. 4-20 illustrate examples graphical user interfaces (GUIs) that may be shown by a processing device that is part of or in operative communication with an ultrasound device. The processing device may be, for example, a mobile phone, tablet, or laptop. The ultrasound device and the processing device may communicate over a wired communication link (e.g., over Ethernet, a Universal Serial Bus (USB) cable or a Lightning cable) or over a wireless communication link (e.g., over a BLUETOOTH, WiFi, or ZIGBEE wireless communication link).

FIG. 4 illustrates an example graphical user interface (GUI) 400 that may be shown by the processing device, in accordance with certain embodiments described herein. The GUI 400 includes an ultrasound image 402, a quality indicator 404, and a share feedback option 424. The quality indicator 404 includes a bar 406 having a first end 408 and a second end 410, an acceptability indicator 420, a slider 418, and text 412.

The ultrasound image 402 may be formed from ultrasound data that was collected by the ultrasound device (not shown in FIG. 4). The ultrasound image 402 may have been displayed by the processing device at the time when a freeze option (not shown) was selected by the user. The ultrasound image 402 may have then been frozen on the display screen of the processing device. The quality indicator 404 may indicate a quality calculated for a sequence of images previously collected with the ultrasound device for performing a particular measurement (e.g., calculating a clinical metric such as ejection fraction). The quality may be calculated by one or more statistical models. The slider 418 may be located at any position along the length of the bar, and the distance of the slider 418 from the first end 408 of the bar 406 relative to the total length of the bar 406 may be proportional to the quality. In FIG. 4, the slider 418 is located near the second end 410 of the bar 406, indicating that the quality may be near to 100% on a scale of 0% to 100%. The acceptability indicator 420 is a black bar located approximately 50% of the distance from the first end 408 to the second end 410 of the bar 406. The acceptability indicator 420 may indicate a threshold quality above which a sequence of images may be considered acceptable or not acceptable for performing the particular measurement. Thus, when the slider 418 is to the left of the acceptability indicator 420, that may indicate that the sequence of images is considered unacceptable for performing the particular measurement. When the slider 418 is to the right of the acceptability indicator 420, that may indicate that the sequence of images is considered acceptable for performing the particular measurement. In FIG. 4, the slider 418 is to the right of the acceptability indicator 420, and thus the sequence of images may be considered acceptable for performing the particular measurement. In FIG. 4, because the quality is above the threshold indicated by the acceptability indicator 420, the slider 418 includes a checkmark symbol that further indicates that the sequence of images is considered acceptable for performing the particular measurement. The quality indicator 404 further includes text 412 indicating whether the sequence of images is considered acceptable for performing the particular measurement. In FIG. 4, the text 412 reads “Measurable,” indicating that based on the calculated quality, the ultrasound image 402 is acceptable for performing the measurement. For further description of the quality indicator 404, see U.S. patent application Ser. No. 16/172,076 titled “QUALITY INDICATORS FOR COLLECTION OF AND AUTOMATED MEASUREMENT ON ULTRASOUND IMAGES,” filed on Oct. 26, 2018 (and assigned to the assignee of the instant application).

In FIG. 4, as well as other embodiments described herein, the bar may be shown as a color scale, with the color changing from the first end to the second end. For example, the first end 408 may be red and the second end 410 green, with the color changing gradually along the length of the bar between the first and second ends. In the black and white representation of FIG. 4, a fill pattern or stippling may be used. For example, red may be represented by dense stippling as shown, yellow by medium stippling, and green by light stippling.

The user may provide feedback about whether the user considers the ultrasound image 402 to be acceptable or unacceptable for performing the measurement by selecting the share feedback option 424. Upon receiving a selection of the share feedback option 424, the processing device may display the GUI 500 or the GUI 600.

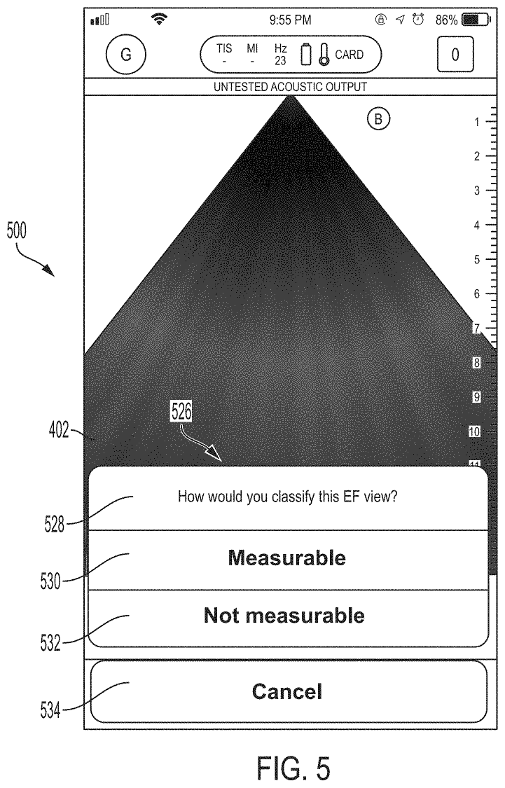

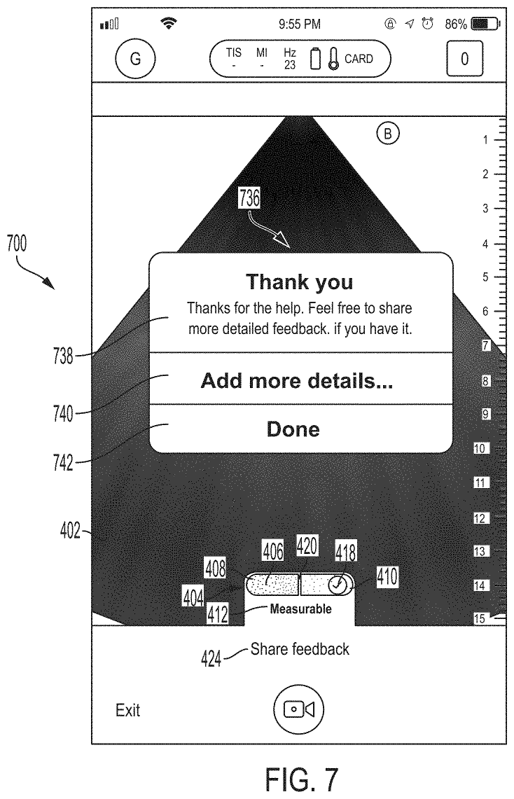

FIG. 5 illustrates an example GUI 500 that may be displayed by the processing device, in accordance with certain embodiments described herein. In FIG. 5, the GUI 500 includes the ultrasound image 402, a dialog box 526, and a cancel option 534. The dialog box 526 (which in this embodiment is superimposed over at least a portion of the ultrasound image 402, although other display locations are also contemplated) includes text 528, a measurable option 530, and a not measurable option 532. The text 528 prompts the user to select either the measurable option 530 or the not measurable option 532 to indicate how the user would classify the ultrasound image 402. If the user considers the ultrasound image 402 acceptable for performing the measurement, the user may select the measurable option 530. If the user does not consider the ultrasound image 402 acceptable for performing the measurement, the user may select the not measurable option 532. The feedback received from the user by the processing device may therefore be selection of either the measurable option 530 or the not measurable option 532. In some embodiments, upon receiving a selection of either the measurable option 530 or the not measurable option 532, the processing device may upload, to the cloud (e.g., one more remote servers), the ultrasound image 402, the quality automatically calculated for the ultrasound image 402 (and shown by the quality indicator 404), and an indication of whether the user selected the measurable option 530 or the not measurable option 532. In some embodiments, the processing device may upload this information later, as will be described below. Additionally, upon receiving a selection of either the measurable option 530 or the not measurable option 532, the processing device may display the GUI 700 as illustrated in FIG. 7. Upon receiving a selection of the cancel option 534, the processing device may begin to display in real-time ultrasound images collected by the ultrasound device, without uploading feedback.

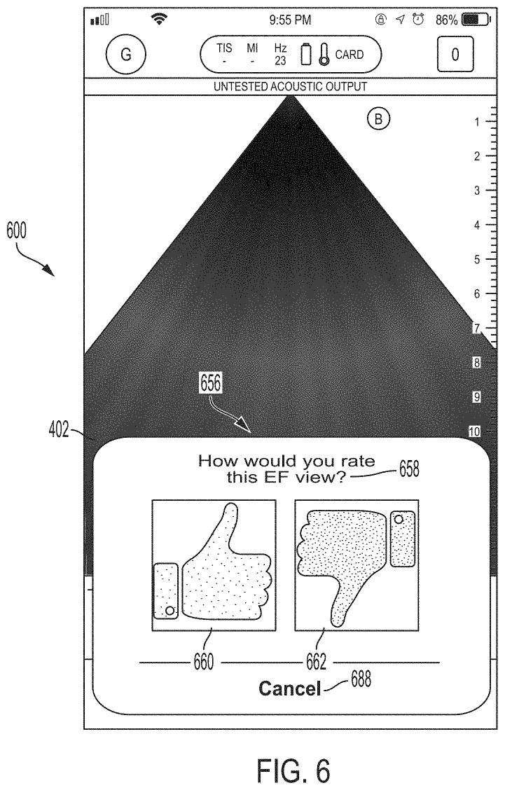

FIG. 6 illustrates another example GUI 600 that may be displayed by the processing device, in accordance with certain embodiments described herein. In some embodiments, the processing device may display the GUI 600 instead of the GUI 500. In FIG. 6, the GUI 600 includes the ultrasound image 402 and a dialog box 656. The dialog box 656 (which in this embodiment is superimposed over at least a portion of the ultrasound image 402, although other display locations are also contemplated) includes text 658, a thumbs up option 660, and a thumbs down option 662. The text 658 prompts the user to either select the “thumbs up” option 660 or the “thumbs down” option 662 in order to rate the acceptability of the ultrasound image 402 for performing a measurement. Selection of the thumbs up option 660 may indicate that the user considers the ultrasound image 402 acceptable for performing the measurement and selection of the thumbs down option 662 may indicate that the user considers the ultrasound image 402 not acceptable for performing the measurement. Optionally, color may also be used to further distinguish between the two options (e.g., green (light stippling) for “thumbs up” and red (dense stippling) for “thumbs down”). The feedback received from the user by the processing device may therefore be selection of either the thumbs up option 660 or the thumbs down option 662. Upon receiving a selection of either the thumbs up option 660 or the thumbs down option 662, the processing device may upload, to the cloud (e.g., one more remote servers), the ultrasound image 402 and an indication of whether the thumbs up option 660 or the thumbs down option 662 was selected. However, in some embodiments, this information may be uploaded later, as will be described below. Additionally, upon receiving a selection of either the thumbs up option 660 or the thumbs down option 662, the processing device may display the GUI 700. Upon receiving a selection of the cancel option 688, the processing device may begin to display in real-time ultrasound images collected by the ultrasound device, without uploading feedback.

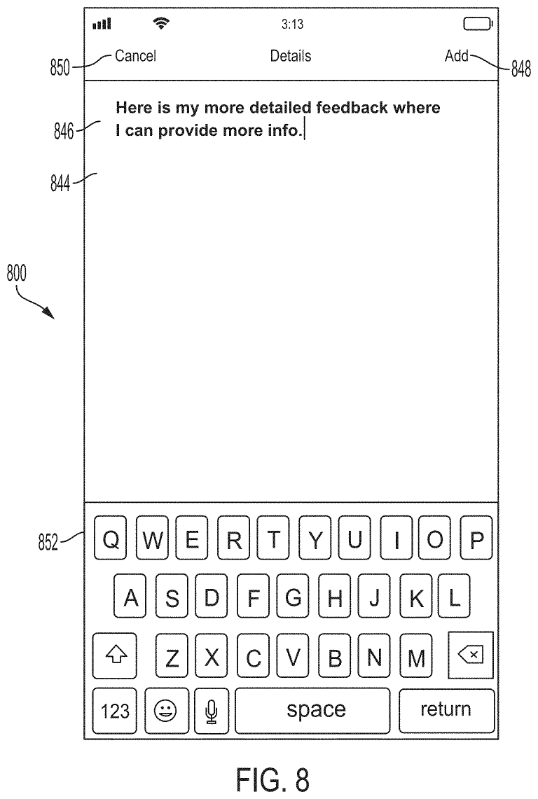

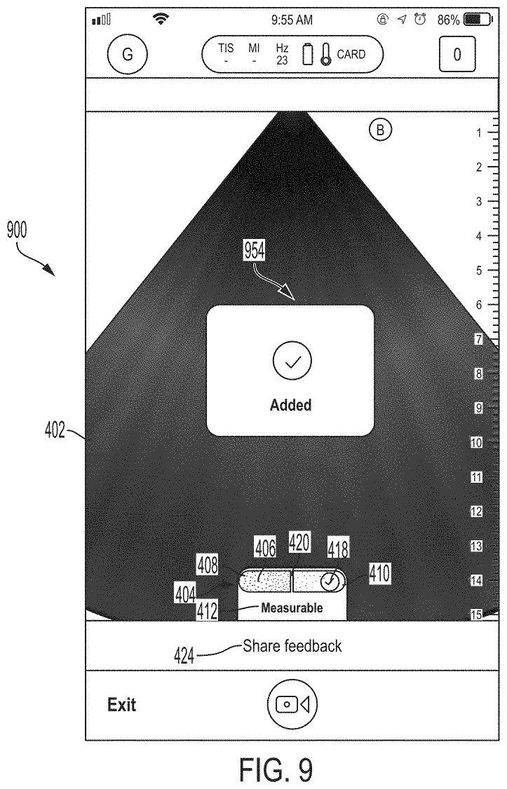

FIG. 7 illustrates an example GUI 700 that may be displayed by the processing device, in accordance with certain embodiments described herein. The GUI 700 includes the ultrasound image 402, the quality indicator 404, and a dialog box 736. The dialog box 736 (which in this embodiment is superimposed over at least a portion of the ultrasound image 402, although other display locations are also contemplated) includes text 738, an add more details option 740, and a done option 742. The text 738 prompts the user to add more detailed feedback regarding the ultrasound image 402. Upon receiving a selection of the add more details option 740, the processing device may display the GUI 800 as illustrated in FIG. 8. Upon receiving a selection of the done option 742, if the processing device previously uploaded the ultrasound image 402, the quality automatically calculated for the ultrasound image 402, and an indication of whether the user selected the measurable option 530 or the not measurable option 532, the processing device may begin to display in real-time ultrasound images collected by the ultrasound device. If the processing device did not previously upload this information, upon receiving a selection of the done option 742, the processing device may upload this information and begin to display in real-time ultrasound images collected by the ultrasound device. In some embodiments, upon receiving a selection of the done option 742, the processing device may also display an indicator (e.g., the indicator 954 of FIG. 9) that indicates that the feedback has been uploaded.

FIG. 8 illustrates an example GUI 800 that may be displayed by the processing device, in accordance with certain embodiments described herein. The GUI 800 includes a text space 844, text 846, an add option 848, a cancel option 850, and a keyboard 852. The user may type the text 846 using the keyboard 852, causing the text 846 to appear in the text space 844 as the text 846 is typed on the keyboard 852. Upon receiving a selection of the add option 848, if the processing device previously uploaded the ultrasound image 402, the quality automatically calculated for the ultrasound image 402, and an indication of whether the user selected the measurable option 530 or the not measurable option 532, the processing device may upload to the cloud the text 846 in the text space 844, to be associated with the ultrasound image 402. If the processing device did not previously upload this information, upon receiving a selection of the add option 848, the processing device may upload this information and the text 846 in the text space 844 to the cloud. Additionally, upon receiving a selection of the add option 848, the processing display may display the GUI 900 as illustrated in FIG. 9. Upon receiving a selection of the cancel option 850, if the processing device did not previously upload to the cloud the ultrasound image 402, the quality automatically calculated for the ultrasound image 402, and an indication of whether the user selected the measurable option 530 or the not measurable option 532, the processing device may upload this information to the cloud. If the processing did previously upload this information to the cloud, the processing device may not upload any further information to the cloud upon receiving a selection of the cancel option 850. Additionally, upon receiving a selection of the cancel option 850, the processing display may begin to display in real-time ultrasound images collected by the ultrasound device.

FIG. 9 illustrates another example of a GUI 900 that may be displayed by the processing device, in accordance with certain embodiments described herein. The GUI 900 includes the ultrasound image 402, the quality indicator 404, the send feedback option 424, and an indicator 954. The indicator 954 indicates that the feedback submitted as text through the GUI 800 has been added.

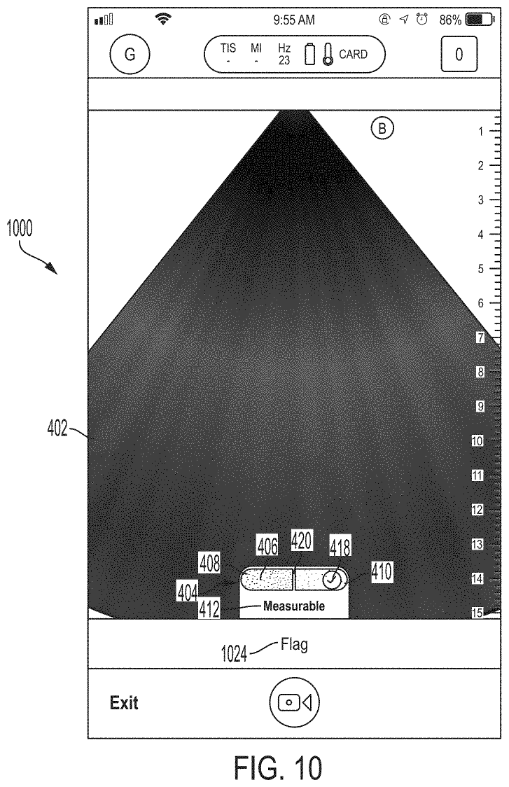

FIG. 10 illustrates another example GUI 1000 that may be shown by the processing device, in accordance with certain embodiments described herein. In some embodiments, the processing device may display the GUI 1000 instead of the GUI 400. The GUI 1000 is the same as the GUI 400 except the GUI 1000 includes the flag option 1024 instead of the share feedback option 424. In some embodiments, upon receiving a selection of the flag option 1024, the processing device may upload, to the cloud (e.g., one more remote servers), the ultrasound image 402, the quality automatically calculated for the ultrasound image 402 (and shown by the quality indicator 404), and an indication that the user selected the flag option 1024. Selection of the flag option 1024 may indicate that the user considers that the ultrasound image 402 and/or the quality should be flagged for review. In some embodiments, the processing device may display both the share feedback option 424 and the flag option 1024.

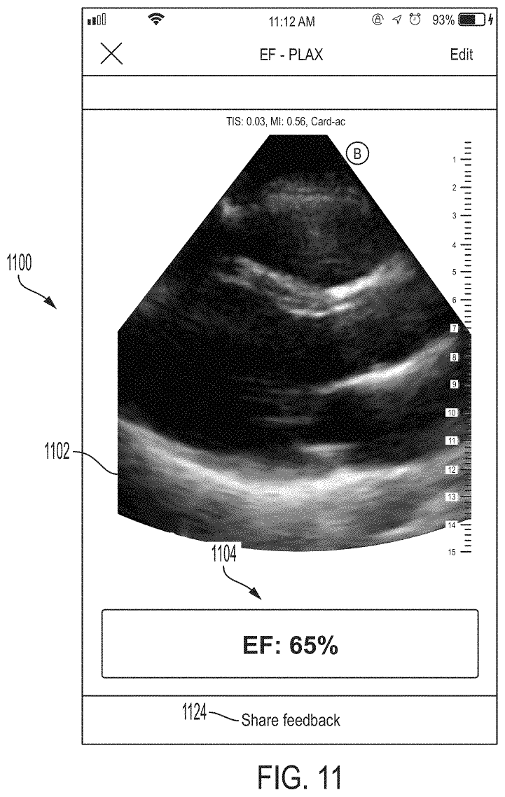

FIG. 11 illustrates another example GUI 1100 that may be shown by the processing device, in accordance with certain embodiments described herein. The GUI 1100 includes an ultrasound image 1102, a measurement value 1104, and a share feedback option 1124. The ultrasound image 1102 may be formed from ultrasound data that was collected by the ultrasound device. The ultrasound image 1102 may have been displayed by the processing device at the time when a freeze option (not shown) was selected by the user. The ultrasound image 1102 may have then been frozen on the display screen of the processing device. The measurement value 1104 indicates a result of a measurement performed automatically based at least in part on the ultrasound image 1102. The measurement value 1104 may be calculated by one or more statistical models. In FIG. 11, the measurement is ejection fraction measured on an ultrasound image 1102 showing the parasternal long axis view of the heart. For further description of automatically performing measurements, see U.S. patent application Ser. No. 15/626,423 titled “AUTOMATIC IMAGE ACQUISITION FOR ASSISTING A USER TO OPERATE AN ULTRASOUND IMAGING DEVICE,” filed on Jun. 19, 2017 (and assigned to the assignee of the instant application).

The user may provide feedback about the user considers to be the correct value for the measurement on the ultrasound image 1102 by selecting the share feedback option 1124. Upon receiving a selection of the share feedback option 1124, the processing device may display the GUI 1200, the GUI 1300, the GUI 1400, the GUI 1500, or the GUI 1600 (as described in further detail below). In some embodiments, there may be a default setting as to which of these GUIs the processing device displays after receiving a selection of the share feedback option 1124. In some embodiments, there may be a user-configured setting as to which of these GUIs the processing device displays after receiving a selection of the share feedback option 1124. In some embodiments, after receiving a selection of the share feedback option 1124, the processing device may display a GUI from which the user may select which of these GUIs to display.

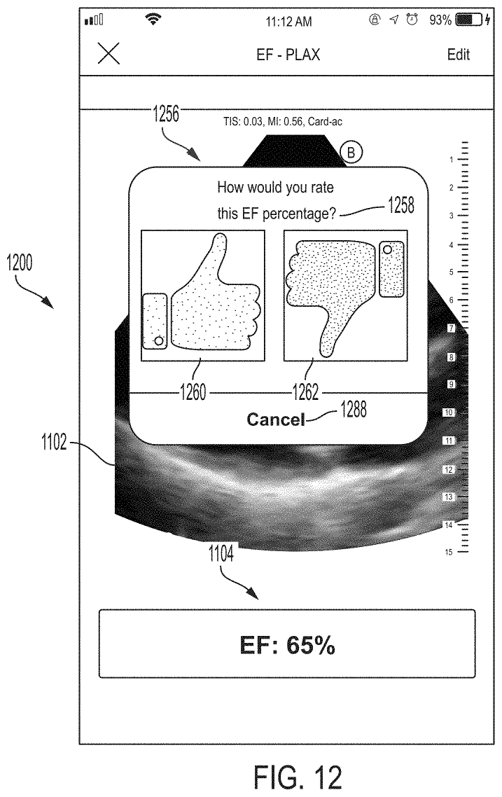

FIG. 12 illustrates another example GUI 1200 that may be displayed by the processing device, in accordance with certain embodiments described herein. In FIG. 12, the GUI 1200 includes the ultrasound image 1102, the measurement value 1104, and a dialog box 1256. The dialog box 1256 (which in this embodiment is superimposed over at least a portion of the ultrasound image 1102, although other display locations are also contemplated) includes text 1258, a thumbs up option 1260, and a thumbs down option 1262. The text 1258 prompts the user to either select the “thumbs up” option 1260 or the “thumbs down” option 1262 in order to rate the measurement value 1104. Selection of the thumbs up option 1260 may indicate agreement with the measurement value 1104 and selection of the thumbs down option 1262 may indicate disagreement with the measurement value 1104. Optionally, color may also be used to further distinguish between the two options (e.g., green for “thumbs up” and red for “thumbs down”). The feedback received from the user by the processing device may therefore be selection of either the thumbs up option 1260 or the thumbs down option 1262. Upon receiving a selection of either the thumbs up option 1260 or the thumbs down option 1262, the processing device may upload, to the cloud (e.g., one more remote servers), the ultrasound image 1102, the measurement value 1104, and an indication of whether the thumbs up option 1260 or the thumbs down option 1262 was selected. However, in some embodiments, this information may be uploaded later, as will be described below. Additionally, upon receiving a selection of either the thumbs up option 1260 or the thumbs down option 1262, the processing device may display the GUI 1700. Upon receiving a selection of the cancel option 1288, the processing device may begin to display in real-time ultrasound images collected by the ultrasound device, without uploading feedback.

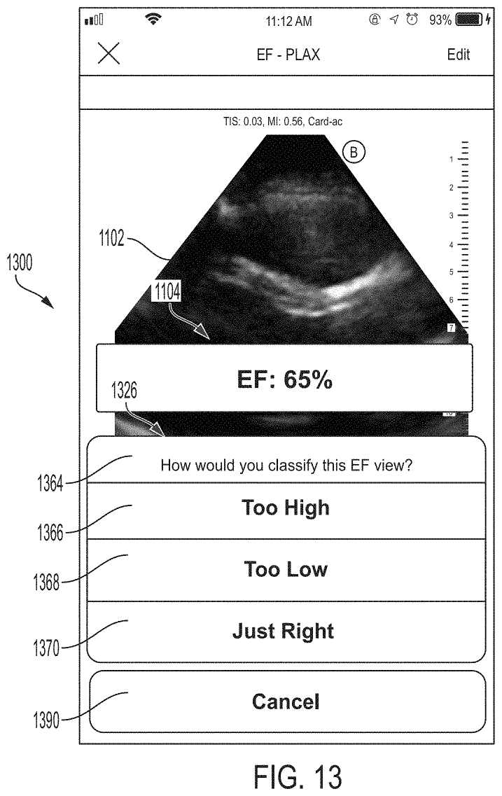

FIG. 13 illustrates another example GUI 1300 that may be displayed by the processing device, in accordance with certain embodiments described herein. In FIG. 13, the GUI 1300 includes the ultrasound image 1102, the measurement value 1104, and a dialog box 1326. The dialog box 1326 (which in this embodiment is superimposed over at least a portion of the ultrasound image 1102, although other display locations are also contemplated) includes text 1364, a too high option 1366, a too low option 1368, a just right option 1370, and a cancel option 1390. The text 1364 prompts the user to select either the too high option 1366, the too low option 1368, or the just right option 1370 in order to classify the measurement value 1104. Selection of the too high option 1366 may indicate that the user considers the measurement value 1104 to be too high, selection of the too low option 1368 may indicate that the user considers the measurement value 1104 to be too low, and selection of the just right option 1370 may indicate that the user considers the measurement value 1104 to be correct. The feedback received from the user by the processing device may therefore be selection of either the too high option 1366, the too low option 1368, or the just right option 1370. Upon receiving a selection of either the too high option 1366, the too low option 1368, or the just right option 1370, the processing device may upload, to the cloud (e.g., one more remote servers), the ultrasound image 1102, the measurement value 1104, and an indication of whether the too high option 1366, the too low option 1368, or the just right option 1370 was selected. However, in some embodiments, this information may be uploaded later. Additionally, upon receiving a selection of either the too high option 1366, the too low option 1368, or the just right option 1370, the processing device may display the GUI 1700. Upon receiving a selection of the cancel option 1390, the processing device may begin to display in real-time ultrasound images collected by the ultrasound device, without uploading feedback.

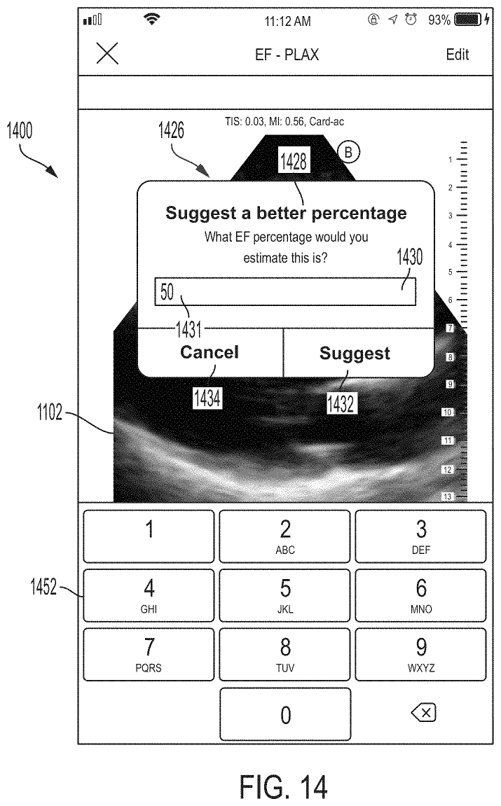

FIG. 14 illustrates another example GUI 1400 that may be displayed by the processing device, in accordance with certain embodiments described herein. In FIG. 14, the GUI 1400 includes the ultrasound image 1102, a dialog box 1426, and a number pad 1452. The dialog box 1426 (which in this embodiment is superimposed over at least a portion of the ultrasound image 1102, although other display locations are also contemplated) includes text 1428, a number space 1430, a value 1431, a suggest option 1432, and a cancel option 1434. The text 1428 prompts the user to input into the text space 1430 a value for the measurement on the ultrasound image 1102 that the user considers to be correct. The user may type the value 1431 using the number pad 1452, causing the value 1431 to appear in the number space 1430. The feedback received from the user by the processing device may therefore be the value 1431 typed in the number space 1430. Upon receiving a selection of the suggest option 1432, the processing device may upload, to the cloud (e.g., one more remote servers), the ultrasound image 1102, the measurement value 1104, and the value 1431 inputted into the number space 1430. However, in some embodiments, this information may be uploaded later. Additionally, upon receiving a selection of the suggest option 1432, the processing device may display the GUI 1700. Upon receiving a selection of the cancel option 1434, the processing device may begin to display in real-time ultrasound images collected by the ultrasound device, without uploading feedback.

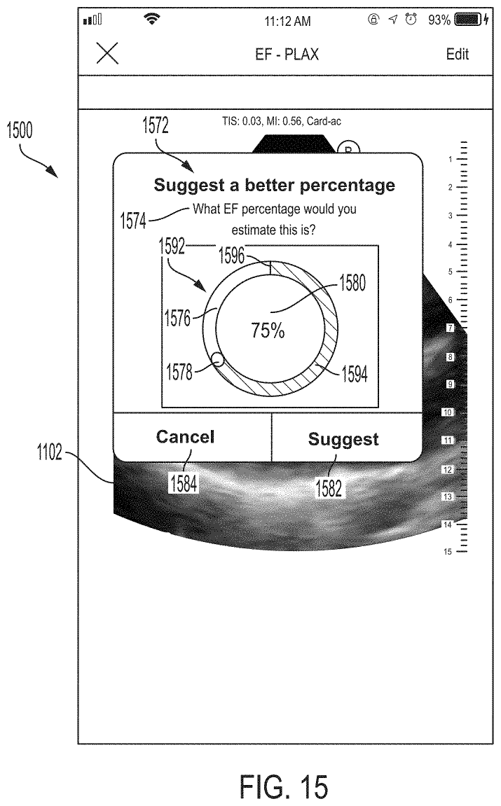

FIG. 15 illustrates another example GUI 1500 that may be displayed by the processing device, in accordance with certain embodiments described herein. In FIG. 15, the GUI 1500 includes the ultrasound image 1102 and a dialog box 1572. The dialog box 1572 (which in this embodiment is superimposed over at least a portion of the ultrasound image 1102, although other display locations are also contemplated) includes text 1574, a slider wheel 1592, a suggest option 1582, and a cancel option 1584. The slider wheel 1592 includes a circle 1576, a slider 1578, a value 1580, a highlighted region 1594, and a starting point 1596. The text 1572 prompts the user to use the slider wheel 1592 to select a value for the measurement on the ultrasound image 1102 that the user considers to be correct. The user my drag the slider 1578 around the circle 1576. The highlighted region 1594 may encompass a portion of the circle 1576 between the starting point 1596 and the slider 1578. The value 1580 may be equal to the portion of the total circumference of the circle 1576 encompassed by the highlighted region 1594. Thus, by dragging the slider 1578 to a particular position along the circle 1576, the user may select a particular value 1580. The feedback received from the user by the processing device may therefore be the value 1580. Upon receiving a selection of the suggest option 1582, the processing device may upload, to the cloud (e.g., one more remote servers), the ultrasound image 1102, the measurement value 1104, and the value 1580. However, in some embodiments, this information may be uploaded later. Additionally, upon receiving a selection of the suggest option 1582, the processing device may display the GUI 1700. Upon receiving a selection of the cancel option 1584, the processing device may begin to display in real-time ultrasound images collected by the ultrasound device, without uploading feedback.

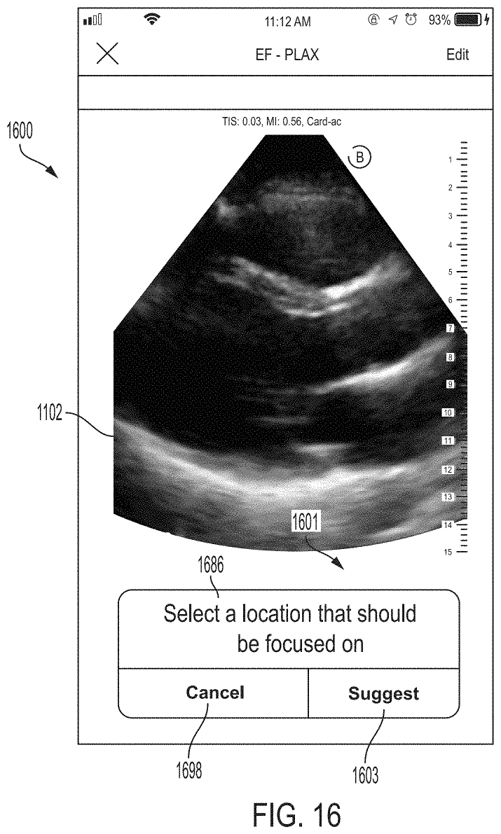

FIG. 16 illustrates another example GUI 1600 that may be displayed by the processing device, in accordance with certain embodiments described herein. In FIG. 16, the GUI 1600 includes the ultrasound image 1102 and a dialog box 1601. The dialog box 1601 (which in this embodiment is superimposed over at least a portion of the ultrasound image 1102, although other display locations are also contemplated) includes text 1686, a suggest option 1603, and a cancel option 1698. The text 1686 prompts the user to select a location on the ultrasound image 1102 where the statistical models should have focused when automatically performing the measurement. For example, calculating ejection fraction may include determining the locations of anatomical keypoints on ultrasound images. The processing device may receive a selection of one or more locations for such anatomical keypoints that the user considers to be correct for performing the measurement. In some embodiments, upon receiving a selection a location on the ultrasound image 1102 (e.g., based on touch or a click), the processing device may display an indication of the location (e.g., crosshairs) on the ultrasound image 1102. In some embodiments, the processing device may display, on a touch-sensitive display screen, the locations of anatomical keypoints on the ultrasound image 1102 used by the statistical models for performing the measurement, detect a dragging movement by the user from the location of one of the anatomical keypoints to a new location, and display the anatomical keypoint in the new location. The feedback received from the user by the processing device may therefore be the locations on the ultrasound image 1102 selected by the user. Upon receiving a selection of the suggest option 1603, the processing device may upload, to the cloud (e.g., one more remote servers), the ultrasound image 1102, the measurement value 1104, and the selected locations. However, in some embodiments, this information may be uploaded later. Additionally, upon receiving a selection of the suggest option 1603, the processing device may display the GUI 1700. Upon receiving a selection of the cancel option 1698, the processing device may begin to display in real-time ultrasound images collected by the ultrasound device, without uploading feedback.

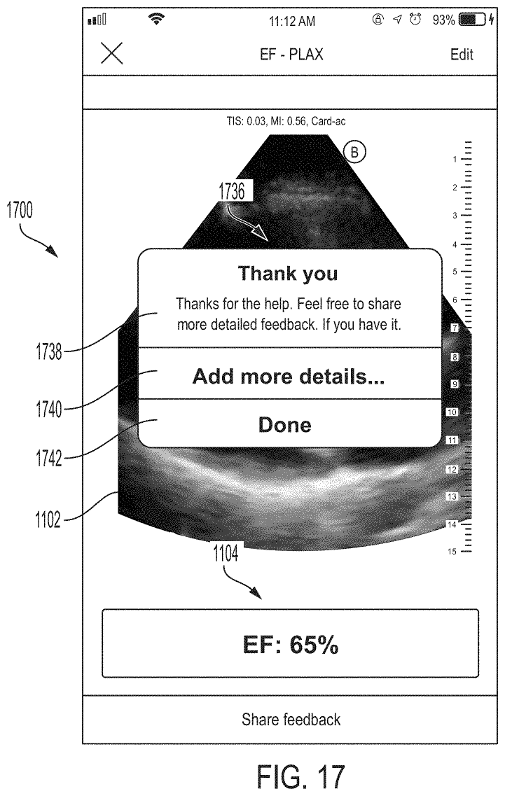

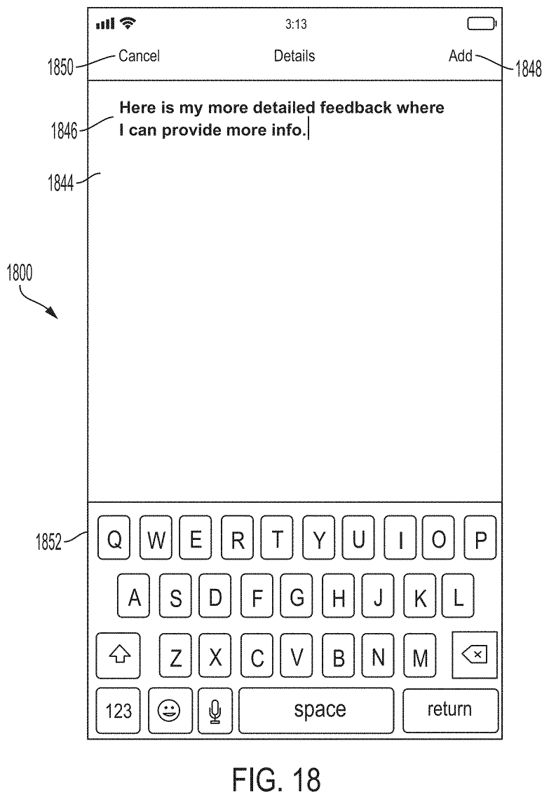

FIG. 17 illustrates an example GUI 1700 that may be displayed by the processing device, in accordance with certain embodiments described herein. The GUI 1700 includes the ultrasound image 1102, the measurement value 1104, and a dialog box 1736. The dialog box 1736 (which in this embodiment is superimposed over at least a portion of the ultrasound image 1102, although other display locations are also contemplated) includes text 1738, an add more details option 1740, and a done option 1742. The text 1738 may prompt the user to add more detailed feedback regarding the ultrasound image 1102. Upon receiving a selection of the add more details option 1740, the processing device may display the GUI 1800 as illustrated in FIG. 18. Upon receiving a selection of the done option 742, if the processing device previously uploaded the ultrasound image 402, measurement value 1104, and the feedback provided by the user, the processing device may begin to display in real-time ultrasound images collected by the ultrasound device. If the processing device did not previously upload this information, upon receiving a selection of the done option 1742, the processing device may upload this information and begin to display in real-time ultrasound images collected by the ultrasound device. In some embodiments, upon receiving a selection of the done option 1742, the processing device may also display an indicator (e.g., the indicator 1954) that indicates that the feedback has been uploaded.

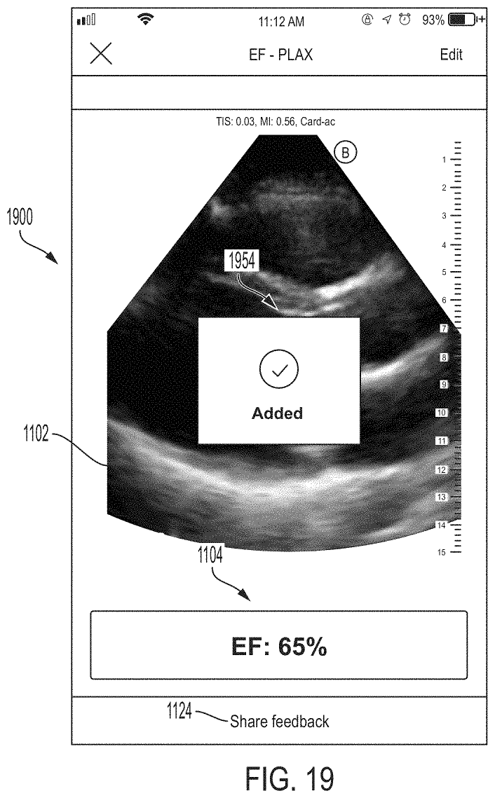

FIG. 18 illustrates an example GUI 1800 that may be displayed by the processing device, in accordance with certain embodiments described herein. The GUI 1800 includes a text space 1844, text 1846, an add option 1848, a cancel option 1850, and a keyboard 1852. The user may type the text 1846, causing it to appear in the text space 1844, using the keyboard 1852. Upon receiving a selection of the add option 1848, if the processing device previously uploaded the ultrasound image 1102, the measurement value 1104, and the feedback provided by the user, the processing device may upload to the cloud the text 1846 in the text space 1844, to be associated with the ultrasound image 1102. If the processing device did not previously upload this information, upon receiving a selection of the add option 1848, the processing device may upload this information in addition to the text 1846 in the text space 1844. Additionally, upon receiving a selection of the add option 1848, the processing display may display the GUI 1900 as illustrated in FIG. 19. Upon receiving a selection of the cancel option 1850, if the processing device did not previously upload to the cloud the ultrasound image 1102, the measurement value 1104, and the feedback provided by the user, the processing device may upload this information to the cloud. If the processing did previously upload this information to the cloud, the processing device may not upload any further information to the cloud upon receiving a selection of the cancel option 1850. Additionally, upon receiving a selection of the cancel option 1850, the processing display may begin to display in real-time ultrasound images collected by the ultrasound device.

FIG. 19 illustrates another example of a GUI 1900 that may be displayed by the processing device, in accordance with certain embodiments described herein. The GUI 1900 includes the ultrasound image 1102, the measurement value 1104, the send feedback option 1124, and an indicator 1954. The indicator 1954 indicates that the feedback submitted as text through the GUI 1900 has been added.

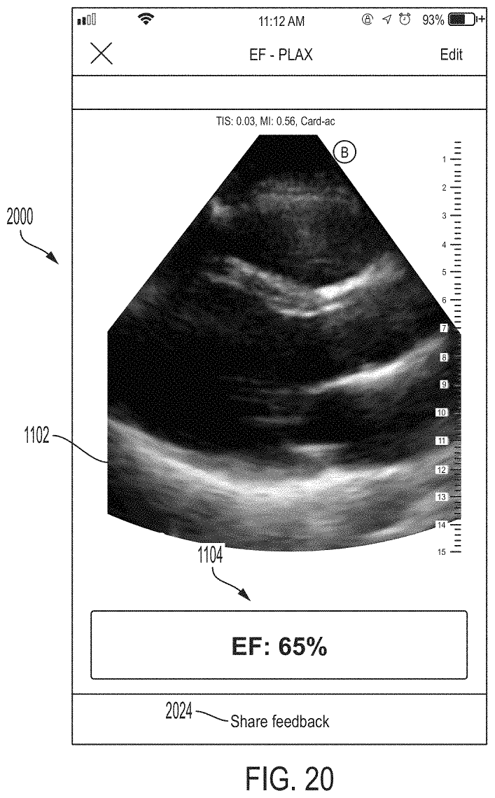

FIG. 20 illustrates another example GUI 2000 that may be shown by the processing device, in accordance with certain embodiments described herein. In some embodiments, the processing device may display the GUI 2000 instead of the GUI 1100. The GUI 2000 is the same as the GUI 1100 except the GUI 2000 includes the flag option 2024 instead of the share feedback option 1124. In some embodiments, upon receiving a selection of the flag option 2024, the processing device may upload, to the cloud (e.g., one more remote servers), the ultrasound image 1102, the measurement value 1104, and an indication that the user selected the flag option 2024. Selection of the flag option 2024 may indicate that the user considers that the ultrasound image 1102 and/or the measurement value 1104 should be flagged for review. In some embodiments, the processing device may display both the share feedback option 1124 and the flag option 2024.

While the above description has described examples of feedback that may be received by the processing device from a user, in some embodiments the processing device may receive other types of feedback. It should be appreciated that the forms of the GUIs shown are non-limiting, and alternative forms may be used. In some embodiments, different texts than the texts shown in the GUIs but which conveys the same or similar meanings may be used. In some embodiments, symbols rather than text may be used. In some embodiments, fewer or additional elements of the GUIs may be shown, or elements of the GUIs may be shown in different relative positions and/or orientations.

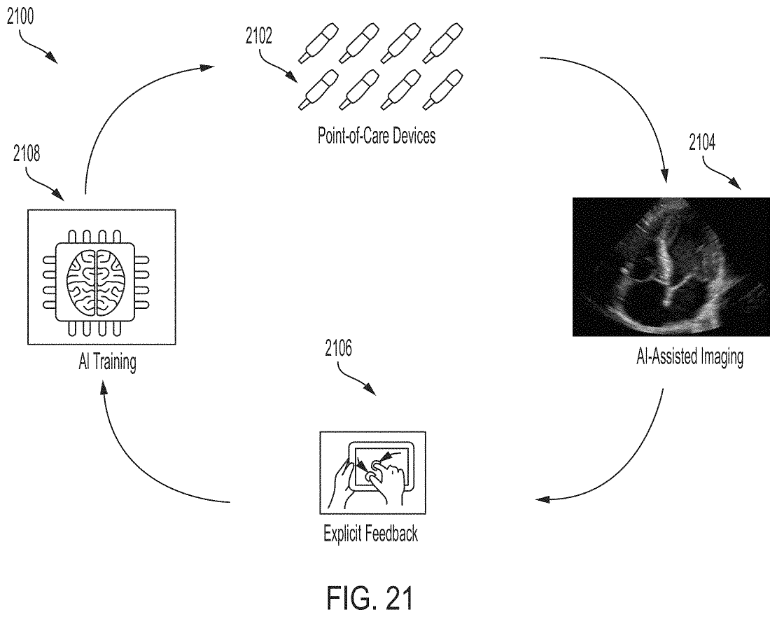

FIG. 21 shows an example of a “virtuous circle” 2100 for continuously improving the performance of certain processes and systems described herein, in accordance with certain embodiments described herein. Stage 2102 shows point-of-care devices (e.g., any of the ultrasound devices discussed previously). Each of the point-of-care devices may be associated with a different individual, group of individuals, institution, or group of institutions, and may be in different locations where care using the plurality of point-of-care devices is administered, such as a patient's home, a medical clinic, a doctor's office, an ambulance, a hospital, etc. Each of the point-of-care devices may include an ultrasound device and a processing device in operative communication with the processing device. Stage 2104 shows that each of the point-of-care devices shown in stage 2102 may be used to perform artificial intelligence (AI)-assisted imaging. For example, the point-of-care devices shown in stage 2102 may collect ultrasound images, and statistical models on the point-of-care devices may perform automatic calculations, such as calculation of qualities of ultrasound images and/or measurement values as described above with reference to the quality indicator 404 and the measurement value 1104. Stage 2106 shows that explicit feedback may be provided by a user based on the ultrasound images and automatic calculations from stage 2104. For example, as described above, the user may provide explicit feedback about whether the user considers an ultrasound image to be acceptable or unacceptable for performing a measurement, and the user's feedback may agree or disagree with the quality calculated by the statistical models. As another example, the user may provide explicit feedback about what the user considers to be the correct value for a measurement on an ultrasound image, and the user's feedback may agree or disagree with the measurement value automatically calculated by the statistical models. The ultrasound images, automatic calculations, and feedback from the user may be uploaded to the cloud (e.g., one or more servers). Stage 2108 shows that the feedback from stage 2106 may be used to train AI models (e.g., convolutional neural networks) to more accurately perform automatic calculations, such as calculations of quality and measurement values. In some embodiments, the AI models may be hosted on the cloud, and the trained models may be downloaded from the cloud to the point-of-care devices shown in stage 2102 (e.g., across a wired or wireless communication link), and used to more accurately perform AI-assisted imaging as shown in stage 2104. The retraining and downloading may occur periodically (e.g., every week, every two weeks, every month, or any other suitable frequency). Accordingly, as more point-of-care devices are deployed, more AI-assisted imaging is performed, more images are produced and analyzed to extract data, and models are trained with more data extracted from images, the point-of-care devices and the AI-assisted imaging they perform may continue to improve as they are updated by the most recent AI training.

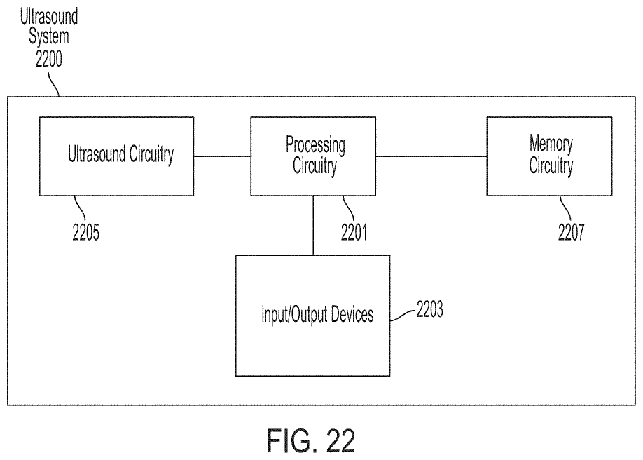

FIG. 22 shows a schematic block diagram illustrating aspects of an example ultrasound system 2200 upon which various aspects of the technology described herein may be practiced. For example, one or more components of the ultrasound system 2200 may perform any of the processes (e.g., the processes 100-300) described herein. As shown, the ultrasound system 2200 includes processing circuitry 2201, input/output devices 2203, ultrasound circuitry 2205, and memory circuitry 2207.

The ultrasound circuitry 2205 may be configured to generate ultrasound data that may be employed to generate an ultrasound image. The ultrasound circuitry 2205 may include one or more ultrasonic transducers monolithically integrated onto a single semiconductor die. The ultrasonic transducers may include, for example, one or more capacitive micromachined ultrasonic transducers (CMUTs), one or more CMOS ultrasonic transducers (CUTs), one or more piezoelectric micromachined ultrasonic transducers (PMUTs), and/or one or more other suitable ultrasonic transducer cells. In some embodiments, the ultrasonic transducers may be formed the same chip as other electronic components in the ultrasound circuitry 2205 (e.g., transmit circuitry, receive circuitry, control circuitry, power management circuitry, and processing circuitry) to form a monolithic ultrasound imaging device.

The processing circuitry 2201 may be configured to perform any of the functionality described herein. The processing circuitry 2201 may include one or more processors (e.g., computer hardware processors). To perform one or more functions, the processing circuitry 2201 may execute one or more processor-executable instructions stored in the memory circuitry 2207. The memory circuitry 2207 may be used for storing programs and data during operation of the ultrasound system 2200. The memory circuitry 2207 may include one or more storage devices such as non-transitory computer-readable storage media. The processing circuitry 2201 may control writing data to and reading data from the memory circuitry 2207 in any suitable manner.

In some embodiments, the processing circuitry 2201 may include specially-programmed and/or special-purpose hardware such as an application-specific integrated circuit (ASIC). For example, the processing circuitry 2201 may include one or more graphics processing units (GPUs) and/or one or more tensor processing units (TPUs). TPUs may be ASICs specifically designed for machine learning (e.g., deep learning). The TPUs may be employed to, for example, accelerate the inference phase of a neural network.

The input/output (I/O) devices 2203 may be configured to facilitate communication with other systems and/or an operator. Example I/O devices 2203 that may facilitate communication with an operator include: a keyboard, a mouse, a trackball, a microphone, a touch screen, a printing device, a display screen, a speaker, and a vibration device. Example I/O devices 2203 that may facilitate communication with other systems include wired and/or wireless communication circuitry such as BLUETOOTH, ZIGBEE, Ethernet, WiFi, and/or USB communication circuitry.

It should be appreciated that the ultrasound system 2200 may be implemented using any number of devices. For example, the components of the ultrasound system 2200 may be integrated into a single device. In another example, the ultrasound circuitry 2205 may be integrated into an ultrasound imaging device that is communicatively coupled with a processing device that includes the processing circuitry 2201, the input/output devices 2203, and the memory circuitry 2207.

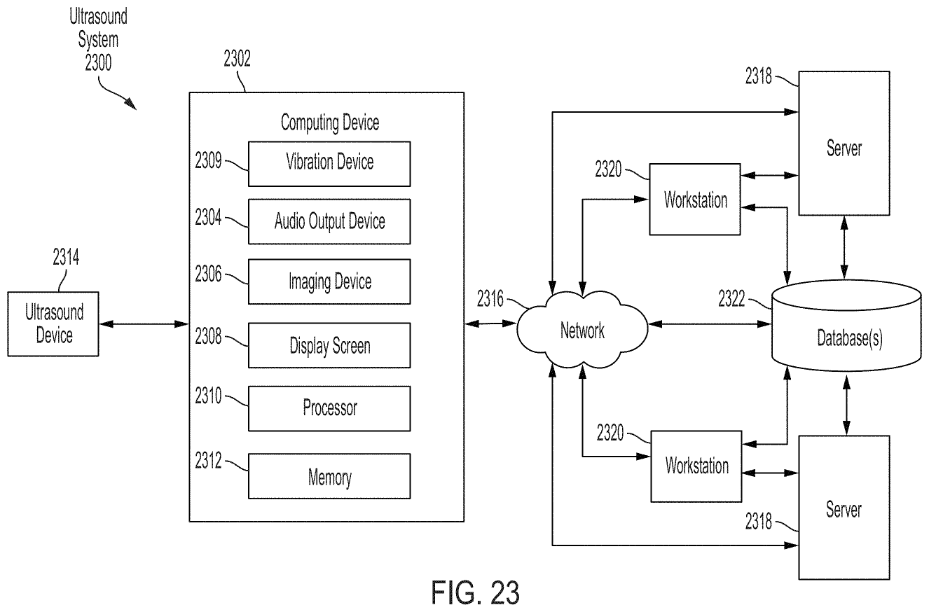

FIG. 23 shows a schematic block diagram illustrating aspects of another example ultrasound system 2300 upon which various aspects of the technology described herein may be practiced. For example, one or more components of the ultrasound system 2300 may perform any of the processes (e.g., the processes 100-300) described herein. As shown, the ultrasound system 2300 includes an ultrasound imaging device 2314 in wired and/or wireless communication with a processing device 2302 (which may correspond to the processing device 400). The processing device 2302 includes an audio output device 2304, an imaging device 2306, a display screen 2308, a processor 2310, a memory 2312, and a vibration device 2309. The processing device 2302 may communicate with one or more external devices over a network 2316. For example, the processing device 2302 may communicate with one or more workstations 2320, servers 2318, and/or databases 2322.

The ultrasound imaging device 2314 may be configured to generate ultrasound data that may be employed to generate an ultrasound image. The ultrasound imaging device 2314 may be constructed in any of a variety of ways. In some embodiments, the ultrasound imaging device 2314 includes a transmitter that transmits a signal to a transmit beamformer which in turn drives transducer elements within a transducer array to emit pulsed ultrasonic signals into a structure, such as a patient. The pulsed ultrasonic signals may be back-scattered from structures in the body, such as blood cells or muscular tissue, to produce echoes that return to the transducer elements. These echoes may then be converted into electrical signals by the transducer elements and the electrical signals are received by a receiver. The electrical signals representing the received echoes are sent to a receive beamformer that outputs ultrasound data.

The processing device 2302 may be configured to process the ultrasound data from the ultrasound imaging device 2314 to generate ultrasound images for display on the display screen 2308. The processing may be performed by, for example, the processor 2310. The processor 2310 may also be adapted to control the acquisition of ultrasound data with the ultrasound imaging device 2314. The ultrasound data may be processed in real-time during a scanning session as the echo signals are received. In some embodiments, the displayed ultrasound image may be updated a rate of at least 5 Hz, at least 10 Hz, at least 20 Hz, at a rate between 5 and 60 Hz, at a rate of more than 20 Hz. For example, ultrasound data may be acquired even as images are being generated based on previously acquired data and while a live ultrasound image is being displayed. As additional ultrasound data is acquired, additional frames or images generated from more-recently acquired ultrasound data are sequentially displayed. Additionally, or alternatively, the ultrasound data may be stored temporarily in a buffer during a scanning session and processed in less than real-time.

Additionally (or alternatively), the processing device 2302 may be configured to perform any of the processes (e.g., the processes 100-300) described herein (e.g., using the processor 2310). As shown, the processing device 2302 may include one or more elements that may be used during the performance of such processes. For example, the processing device 2302 may include one or more processors 2310 (e.g., computer hardware processors) and one or more articles of manufacture that include non-transitory computer-readable storage media such as the memory 2312. The processor 2310 may control writing data to and reading data from the memory 2312 in any suitable manner. To perform any of the functionality described herein, the processor 2310 may execute one or more processor-executable instructions stored in one or more non-transitory computer-readable storage media (e.g., the memory 2312), which may serve as non-transitory computer-readable storage media storing processor-executable instructions for execution by the processor 2310.

In some embodiments, the processing device 2302 may include one or more input and/or output devices such as the audio output device 2304, the imaging device 2306, the display screen 2308, and the vibration device 2309. The audio output device 2304 may be a device that is configured to emit audible sound such as a speaker. The imaging device 2306 may be configured to detect light (e.g., visible light) to form an image such as a camera. The display screen 2308 may be configured to display images and/or videos such as a liquid crystal display (LCD), a plasma display, and/or an organic light emitting diode (OLED) display. The vibration device 2309 may be configured to vibrate one or more components of the processing device 2302 to provide tactile feedback. These input and/or output devices may be communicatively coupled to the processor 2310 and/or under the control of the processor 2310. The processor 2310 may control these devices in accordance with a process being executed by the process 2310 (such as the processes 100-300). Similarly, the processor 2310 may control the audio output device 2304 to issue audible instructions and/or control the vibration device 2309 to change an intensity of tactile feedback (e.g., vibration) to issue tactile instructions. Additionally (or alternatively), the processor 2310 may control the imaging device 2306 to capture non-acoustic images of the ultrasound imaging device 2314 being used on a subject to provide an operator of the ultrasound imaging device 2314 an augmented reality interface.

It should be appreciated that the processing device 2302 may be implemented in any of a variety of ways. For example, the processing device 2302 may be implemented as a handheld device such as a mobile smartphone or a tablet. Thereby, an operator of the ultrasound imaging device 2314 may be able to operate the ultrasound imaging device 2314 with one hand and hold the processing device 2302 with another hand. In other examples, the processing device 2302 may be implemented as a portable device that is not a handheld device such as a laptop. In yet other examples, the processing device 2302 may be implemented as a stationary device such as a desktop computer.

In some embodiments, the processing device 2302 may communicate with one or more external devices via the network 2316. The processing device 2302 may be connected to the network 2316 over a wired connection (e.g., via an Ethernet cable) and/or a wireless connection (e.g., over a WiFi network). As shown in FIG. 23, these external devices may include servers 2318, workstations 2320, and/or databases 2322. The processing device 2302 may communicate with these devices to, for example, off-load computationally intensive tasks. For example, the processing device 2302 may send an ultrasound image over the network 2316 to the server 2318 for analysis (e.g., to identify an anatomical feature in the ultrasound) and receive the results of the analysis from the server 2318. Additionally (or alternatively), the processing device 2302 may communicate with these devices to access information that is not available locally and/or update a central information repository. For example, the processing device 2302 may access the medical records of a subject being imaged with the ultrasound imaging device 2314 from a file stored in the database 2322. In this example, the processing device 2302 may also provide one or more captured ultrasound images of the subject to the database 2322 to add to the medical record of the subject. For further description of ultrasound imaging devices and systems, see U.S. patent application Ser. No. 15/415,434 titled “UNIVERSAL ULTRASOUND IMAGING DEVICE AND RELATED APPARATUS AND METHODS,” filed on Jan. 25, 2017 (and assigned to the assignee of the instant application), which is incorporated by reference herein in its entirety.

Aspects of the technology described herein relate to the application of automated image processing techniques to analyze images, such as ultrasound images or optical images. In some embodiments, the automated image processing techniques may include machine learning techniques such as deep learning techniques. Machine learning techniques may include techniques that seek to identify patterns in a set of data points and use the identified patterns to make predictions for new data points. These machine learning techniques may involve training (and/or building) a model using a training data set to make such predictions. The trained model may be used as, for example, a classifier that is configured to receive a data point as an input and provide an indication of a class to which the data point likely belongs as an output, a segmentation model that is configured to segment areas in an image, or a keypoint localization model configured to find specific keypoints in an image.

Deep learning techniques may include those machine learning techniques that employ neural networks to make predictions. Neural networks typically include a collection of neural units (referred to as neurons) that each may be configured to receive one or more inputs and provide an output that is a function of the input. For example, the neuron may sum the inputs and apply a transfer function (sometimes referred to as an “activation function”) to the summed inputs to generate the output. The neuron may apply a weight to each input, for example, to weight some inputs higher than others. Example transfer functions that may be employed include step functions, piecewise linear functions, and sigmoid functions. These neurons may be organized into a plurality of sequential layers that each include one or more neurons. The plurality of sequential layers may include an input layer that receives the input data for the neural network, an output layer that provides the output data for the neural network, and one or more hidden layers connected between the input and output layers. Each neuron in a hidden layer may receive inputs from one or more neurons in a previous layer (such as the input layer) and provide an output to one or more neurons in a subsequent layer (such as an output layer).

A neural network may be trained using, for example, labeled training data. The labeled training data may include a set of example inputs and an answer associated with each input. For example, the training data may include a plurality of ultrasound images or sets of raw acoustical data that are each labeled (e.g., labeled with classes, segmented areas, or locations of keypoints). In this example, the ultrasound images may be provided to the neural network to obtain outputs that may be compared with the labels associated with each of the ultrasound images. One or more characteristics of the neural network (such as the interconnections between neurons (referred to as edges) in different layers and/or the weights associated with the edges) may be adjusted until the neural network correctly classifies most (or all) of the input images, correctly segments areas in most (or all) of the input images, or correctly finds specific keypoints in most (or all) of the input images.

Once the training data has been created, the training data may be loaded to a database (e.g., an image database) and used to train a neural network using deep learning techniques. Once the neural network has been trained, the trained neural network may be deployed to one or more processing devices. It should be appreciated that the neural network may be trained with any number of sample patient images, although it will be appreciated that the more sample images used, the more robust the trained model data may be.

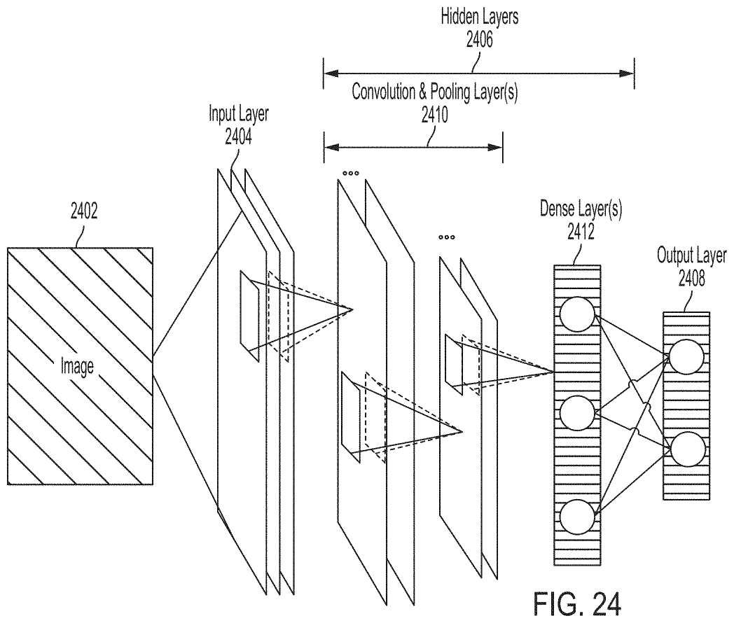

In some applications, a neural network may be implemented using one or more convolution layers to form a convolutional neural network. An example convolutional neural network is shown in FIG. 24 that is configured to analyze an image 2402. As shown, the convolutional neural network includes an input layer 2404 to receive the image 2402, an output layer 2408 to provide the output, and a plurality of hidden layers 2406 connected between the input layer 2404 and the output layer 2408. The plurality of hidden layers 2406 includes convolution and pooling layers 2410 and dense (e.g., fully connected) layers 2412.

The input layer 2404 may receive the input to the convolutional neural network. As shown in FIG. 24, the input the convolutional neural network may be the image 2402. The image 2402 may be, for example, an ultrasound image.

The input layer 2404 may be followed by one or more convolution and pooling layers 2410. A convolutional layer may include a set of filters that are spatially smaller (e.g., have a smaller width and/or height) than the input to the convolutional layer (e.g., the image 2402). Each of the filters may be convolved with the input to the convolutional layer to produce an activation map (e.g., a 2-dimensional activation map) indicative of the responses of that filter at every spatial position. The convolutional layer may be followed by a pooling layer that down-samples the output of a convolutional layer to reduce its dimensions. The pooling layer may use any of a variety of pooling techniques such as max pooling and/or global average pooling. In some embodiments, the down-sampling may be performed by the convolution layer itself (e.g., without a pooling layer) using striding.

The convolution and pooling layers 2410 may be followed by dense layers 2412. The dense layers 2412 may include one or more layers each with one or more neurons that receives an input from a previous layer (e.g., a convolutional or pooling layer) and provides an output to a subsequent layer (e.g., the output layer 2408). The dense layers 2412 may be described as “dense” because each of the neurons in a given layer may receive an input from each neuron in a previous layer and provide an output to each neuron in a subsequent layer. The dense layers 2412 may be followed by an output layer 2408 that provides the output of the convolutional neural network. The output may be, for example, an indication of which class, from a set of classes, the image 2402 (or any portion of the image 2402) belongs to; indications of locations of segmented areas in the image 2402; or indications of locations of keypoints in the image 2402.

It should be appreciated that the convolutional neural network shown in FIG. 24 is only one example implementation and that other implementations may be employed. For example, one or more layers may be added to or removed from the convolutional neural network shown in FIG. 24. Additional example layers that may be added to the convolutional neural network include: a rectified linear units (ReLU) layer, a pad layer, a concatenate layer, and an upscale layer. An upscale layer may be configured to upsample the input to the layer. An ReLU layer may be configured to apply a rectifier (sometimes referred to as a ramp function) as a transfer function to the input. A pad layer may be configured to change the size of the input to the layer by padding one or more dimensions of the input. A concatenate layer may be configured to combine multiple inputs (e.g., combine inputs from multiple layers) into a single output.

For further description of deep learning techniques, see U.S. patent application Ser. No. 15/626,423 titled “AUTOMATIC IMAGE ACQUISITION FOR ASSISTING A USER TO OPERATE AN ULTRASOUND IMAGING DEVICE,” filed on Jun. 19, 2017 (and assigned to the assignee of the instant application). In any of the embodiments described herein, instead of/in addition to using a convolutional neural network, a fully connected neural network may be used. Additionally, while processing of ultrasound images using deep learning techniques is described with reference to FIG. 24, the description may apply equally to processing of optical images.

Various aspects of the present disclosure may be used alone, in combination, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Various inventive concepts may be embodied as one or more processes, of which examples have been provided. The acts performed as part of each process may be ordered in any suitable way. Thus, embodiments may be constructed in which acts are performed in an order different than illustrated, which may include performing some acts simultaneously, even though shown as sequential acts in illustrative embodiments. Further, one or more of the processes may be combined and/or omitted, and one or more of the processes may include additional steps.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

As used herein, reference to a numerical value being between two endpoints should be understood to encompass the situation in which the numerical value can assume either of the endpoints. For example, stating that a characteristic has a value between A and B, or between approximately A and B, should be understood to mean that the indicated range is inclusive of the endpoints A and B unless otherwise noted.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having described above several aspects of at least one embodiment, it is to be appreciated various alterations, modifications, and improvements will readily occur to those skilled in the art. Such alterations, modifications, and improvements are intended to be object of this disclosure. Accordingly, the foregoing description and drawings are by way of example only.

What is claimed is:

1. An apparatus, comprising:
a processing device in operative communication with an ultrasound device and configured to:
receive feedback from a user regarding an automatic calculation performed based on ultrasound data.

2. The apparatus of claim 1, wherein the processing device is further configured to:
perform the automatic calculation based on the ultrasound data.

3. The apparatus of claim 2, wherein the processing device is configured, when performing the automatic calculation, to use one or more statistical models.

4. The apparatus of claim 1, wherein the processing device is further configured to:
receive the ultrasound data from the ultrasound device.

5. The apparatus of claim 1, wherein the automatic calculation based on the ultrasound data comprises a result for a measurement performed automatically on the ultrasound data.

6. The apparatus of claim 5, wherein the processing device is configured, when receiving the feedback from the user, to receive an indication of agreement or disagreement with the result of the measurement.

7. The apparatus of claim 5, wherein the processing device is configured, when receiving the feedback from the user, to receive an indication of whether the result of the measurement is too high, too low, or correct.

8. The apparatus of claim 5, wherein the processing device is configured, when receiving the feedback from the user, to receive a value for the measurement that the user considers to be correct.

9. The apparatus of claim 5, wherein the processing device is configured, when receiving the feedback from the user, to receive one or more locations on one or more ultrasound images where one or more statistical models should have focused when performing the measurement.

10. The apparatus of claim 5, wherein the processing device is configured, when receiving the feedback from the user, to receive a flag to review the ultrasound data and/or the result of the measurement performed automatically.

11. The apparatus of claim 1, wherein the automatic calculation based on the ultrasound data comprises a quality of the ultrasound data determined automatically for performing a measurement on the ultrasound data.

12. The apparatus of claim 11, wherein the processing device is configured, when receiving the feedback from the user, to receive an indication whether the user considers the ultrasound data acceptable for performing the measurement or not.

13. The apparatus of claim 11, wherein the processing device is configured, when receiving the feedback from the user, to receive a flag to review the ultrasound data and/or the quality of the ultrasound data determined automatically.

14. The apparatus of claim 1, wherein the processing device is configured, when receiving the feedback, to receive text from the user.

15. The apparatus of claim 1, wherein the processing device is further configured to:
provide an option that the user may select to provide the feedback.

16. The apparatus of claim 1, wherein the processing device is further configured to display an image produced from the ultrasound data.

17. The apparatus of claim 1, wherein the processing device is further configured to:
upload the ultrasound data used for the automatic calculation, the automatic calculation, and the feedback to one or more servers.

18. The apparatus of claim 17, wherein the one or more servers are configured to train one or more statistical models to more accurately perform the automatic calculation based on the ultrasound data used for the automatic calculation, the automatic calculation, and the feedback.

19. The apparatus of claim 18, wherein the processing device is further configured to download the one or more statistical models from the one or more servers.

20. The apparatus of claim 1, wherein the processing device is further configured to train one or more statistical models to more accurately perform the automatic calculation based on the ultrasound data used for the automatic calculation, the automatic calculation, and the feedback.
